(19) United States
(12) Patent Application Publication
(10) Pub. No.: US 2025/0380982 A1
(43) Pub. Date: Dec. 18, 2025

(54) CATHETER FOR THE LOCAL TREATMENT OF TUMOUR TISSUE IN INTRAVASCULAR AND INTRALUMINAL SPACES

(71) Applicant: RHEINISCH-WESTFÄLISCHE TECHNISCHE HOCHSCHULE (RWTH) AACHEN, Aachen (DE)

(72) Inventors: Joachim Georg PFEFFER, Aachen (DE); Andreas RITTER, Aachen (DE); Federico PEDERSOLI, HL Maastricht (NL); Peter ISFORT, Herzogenrath (DE); Philipp BRUNERS, Aachen (DE)

(21) Appl. No.: 18/877,446

(22) PCT Filed: Jun. 22, 2023
(86) PCT No.: PCT/EP2023/066994

§ 371 Date: Dec. 20, 2024

(30) Foreign Application Priority Data
Jun. 22, 2022 (DE) 10 2022 115 568.4

(51) Int. Cl. A61B 18/14; A61B 18/00

(52) U.S. Cl. CPC A61B 18/1492; A61B 2018/00071; A61B 2018/0022; A61B 2018/00613; A61B 2018/00767; A61B 2018/00982

(57) ABSTRACT

The invention relates to a catheter for locally treating tissue, in particular tumour tissue or other undesirable tissue types, in intravascular and intraluminal spaces for insertion into a vessel for the duration of treatment. The catheter comprises a flexible, cylindrical body (2) having a first lumen (14.1) for receiving a guidewire for guiding the catheter (1), a second lumen (14.2), and at least one third lumen (14.3) for separately introducing chemical and/or biological therapeutic agents into the tissue, wherein the cylindrical body (2) has radio-paque features. The catheter further comprises a conductive structure (6) for generating pulsed electric fields, wherein the conductive structure (6) comprises at least a first and a second structural element (8.1, 8.2). In addition, a first and a second electrical supply line (10.1, 10.2) are provided and insulated from each other. The invention further relates to a catheter system.

The invention relates to a catheter for inserting into a vessel for the duration of treatment for locally treating tissue, in particular tumour tissue or other undesirable tissue types, in intravascular and intraluminal spaces.

The area of application of the catheter is therefore not limited to tumour tissue. The catheter may be used to treat all types of tissue, including tissue that is not necessarily malignant (tumorous) but forms in undesired areas.

Local tumour therapy traditionally involves surgical removal of the tumour. However, surgical removal is often not possible, particularly in the case of tumours growing along existing structures or around vessels, as there is a risk of damaging adjacent structures. These include, for example, tumours growing along bile ducts or at bile duct bifurcations.

Other forms of local therapy, including various thermal and non-thermal ablation methods, are therefore known from the state of the art. Thermal ablation methods include radiofrequency ablation, microwave ablation, and cryoablation. Among the non-thermal ablation methods, electroporation-based methods are particularly noteworthy.

Electroporation describes the effect that pores form in cell membranes as a result of pulsed electrical fields. In the case of irreversible electroporation, the cell membrane is irreversibly damaged, which ultimately leads to the death of the cells. In reversible electroporation, the formation of pores in the cell membranes is reversible. Electrochemotherapy utilises this principle by using the pores (and thus targeting the area of reversible electroporation) to significantly increase the diffusion of chemotherapeutic agents into the cell interior. Calcium electroporation is another therapeutic procedure for treating tissue, particularly tumour tissue, which makes use of the principle of reversible electroporation. Calcium is introduced into the cells by electroporation. Calcium electroporation has been shown to be a safe and effective therapeutic procedure for treating tumour tissue (see Stine Frandsen, Mille Vissing, and Julie Gehl. “A Comprehensive Review of Calcium Electroporation—A Novel Cancer Treatment Modality”. Cancers 2020, 12, 290).

The advantage of these non-thermal ablation procedures in contrast to thermal ablation procedures is that the structure of vessels running directly next to the tumour can be preserved.

WO 2006/104934 A2 relates to a device and a method for ablating cells. The device comprises a probe and an elongated sleeve. Electrodes for ablating the tissue using radio frequency or microwave energy are located at the distal end of the probe. In addition, the device comprises a vacuum source coupled to a proximal end of the elongated sleeve, by means of which a vacuum may be generated to draw the tissue to the probe.

The disadvantage of ablation using radio frequency or microwave energy is that, in addition to the unhealthy tissue, neighbouring anatomical structures can also be thermally damaged and thus irreversibly destroyed. Therefore, ablation using radio frequency or microwave energy is not suitable for tumours growing directly along existing structures.

US2020/0405384 A1 relates to a device for treating lung tumours, comprising an ablation catheter having a radiofrequency electrode. US2020/0405384 A1 thus also describes ablation of the tumour tissue by means of radio frequency energy, wherein the radio frequency energy is associated with the disadvantages mentioned.

WO 2014/039320 A1 relates to a device for the ablation and electroporation of tissue cells. The document discloses a catheter having a flexible body comprising electrodes disposed on a distal segment of the body for delivering energy to the target tissue. The device is configured so that the electrodes can be used to generate a voltage at which electroporation, heat ablation, or a combination of electroporation and heat ablation occurs. The document thus discloses a combination device for different ablation methods.

WO 2013/059913 A1 also relates to a device for different ablation methods. The intravascular ablation device comprises an elongated probe with a balloon at the tip, which can be used for cryogenic treatment, and a radio frequency or electroporation element. In addition, WO 2013/059913 A1 discloses that electroporation may be used to introduce active substances into tissue. However, WO 2013/059913 A1 does not disclose any possibility of introducing active substances separately into cell tissue.

The object of the invention is to provide a device and a method enabling the local and separate introduction of physical, chemical, and/or biological therapeutic agents into intravascular and intraluminal bodies for treating tumour tissue without destroying the surrounding anatomy.

For a catheter of the type indicated above, the object is achieved by a flexible, cylindrical body having a first lumen for receiving a guide wire for controlling the catheter, a second lumen, and at least one third lumen for separately introducing chemical and/or biological therapeutic agents into the tissue. The catheter has radiopaque features so that the catheter is clearly recognisable during image-guided guidance.

The catheter also has a conductive structure for generating pulsed electric fields. The conductive structure comprises at least a first and a second structural element and extends along a distal end segment of the cylindrical body.

In addition, the catheter has a first electrical supply line for connecting the first structural element to an energy source and a second supply line for connecting the second structural element to the energy source. The first and second supply lines are insulated from each other. It is also preferable that the first electrical supply line connects the first structural element to a first energy source, and that the second supply line connects the second structural element to a second energy source. The first energy source and the second energy source are preferably independent of each other.

The energy source is configured to provide different voltages, currents, a variable pulse duration and frequency, as well as a variable number of pulses. The electrical supply lines are made of any electrically conductive material such as stainless steel, copper, gold, silver or conductive plastic, and connect the energy source to the structural elements of the conductive structure so that the structural elements can generate pulsed electrical fields. The energy source is preferably configured to provide a first potential at the first structural element and to provide a second potential at the second structural element, wherein the first potential is higher or lower than the second potential. The energy source is preferably an electrical generator. It is also preferred that the energy source is an electrical generator approved for clinical use. The preferred energy source is a Cliniporator® from IGEA or an AngioDynamics NanoKnife.

The catheter enables treating tissue, particularly tumours, both in intravascular and intraluminal spaces, i.e. both in vascular structures and in cavities in the human body. The catheter-based solution allows for targeted treatment of the tumour, as the flexible body of the catheter can be guided directly to the target lesion via the guide wire. The catheter is not designed to remain in the human body permanently, but is specifically designed to be inserted into the human body only for the duration of the treatment.

The invention utilises the knowledge that electrical fields can be used to physically influence tissue. In order to achieve the desired treatment effect without irreversibly damaging neighbouring structures, the properties of the pulsed electric field must be selected to a suitable degree. The target tissue is typically tumour tissue and therefore pathological tissue, and has electrical properties differing from the electrical properties of the surrounding healthy tissue. Taking into account the specific electrical properties, i.e. the permittivity and the electrical conductivity of the tumour tissue and the adjacent tissue, the voltage, the current intensity, the pulse duration, the pulse frequency, and the number of pulses of the electric field can be adjusted so that pore formation preferably occurs exclusively in the target tissue and the adjacent structures remain undamaged. The catheter therefore allows for treating tumours in the immediate vicinity of large blood vessels.

In addition, the invention allows for introducing chemical and/or biological therapeutic agents into the tumour tissue, so that the catheter thus provides a comprehensive treatment approach.

It is preferable that the second lumen and/or the third lumen is/are configured to introduce calcium. It is further, or alternatively, preferred that the second lumen and/or the third lumen is/are configured to introduce a cytostatic agent, a saline solution, an antibiotic, an immunosuppressive agent, and/or a further agent for bringing about cell death into the tumour tissue, wherein it is preferred that the second lumen and the third lumen are configured to introduce different therapeutic agents mentioned above into the tumour tissue. However, it is also possible that the second lumen and the third lumen are configured to introduce the same therapeutic agent into the tumour tissue, but in different quantities and/or at different locations. For example, it is preferred that the second lumen introduces calcium into a first tissue segment of the tissue to be treated, and that the second lumen introduces calcium into a second tissue segment of the tissue to be treated.

The flexible, cylindrical body of the catheter is preferably made of a thermoplastic, elastomer, and/or silicone, wherein the plastic is preferably a medical-grade plastic, particularly preferably a solution-grade plastic. In addition, the body is preferably configured to be torsionally stable and tension-resistant.

It is also preferred that the catheter has more than three lumens, for example four, five, six, seven lumens, or more than seven lumens. The additional lumens may be provided for feeding further therapeutic agents into the tissue, for supplying and removing rinsing solutions, or for conveying pressurised air and other fluids.

Furthermore, it is preferred that the conductive structure comprises more than two structural elements, for example three, four, five, six, seven, or more than seven structural elements, wherein the structural elements are preferably disposed at equal distances from each other. This has the advantage that electric fields having different properties can be generated or the therapeutically effective part of the field propagation can be adapted more precisely to the volume of the target tissue. It is preferable in this respect that the energy source is configured to provide different potentials at the first, the second and/or the other structural elements. In this way, different voltages and thus different fields can be generated between the structural elements. For example, a field may be generated between the first structural element and the second structural element having a lower or higher field strength than a field generated between the second structural element and a third structural element. In this respect, the field distribution of the fields generated between the structural elements can be adapted to the tumour tissue to be treated. For example, a stronger field can be generated on a segment of tumour tissue showing increased deep growth than on a segment of merely superficial tumour tissue. Positioning errors or inaccuracies of the electrodes may also be compensated by means of said method. The generated electric field may also depend on the tissue-specific electrical properties of the tumour tissue.

The conductive structure is preferably disposed on an outer surface of the cylindrical body. The advantage of said arrangement is that the conductive structure may press partially or completely into the tumour tissue, or, if the tumour is growing around a vessel, into the vessel.

Alternatively, the conductive structure is preferably disposed on an inner side of the cylindrical body. The advantage of said arrangement is that the catheter has an outer side without structural elements and therefore a uniformly round shape on the outside. This results in improved handling and guidance of the catheter. Furthermore, the manufacture of the catheter including the conductive structure is simplified and the catheter is more stable overall. It should be noted that preferably no insulating layer is disposed between the conductive structure and the tumour tissue, or, in the event that the tumour tissue grows around a vessel, between the conductive structure and the vessel, so that conductivity to the tumour tissue to be treated is ensured.

In a further, preferred embodiment, the conductive structure is disposed both on the outside and on the inside of the cylindrical catheter body. This can also improve the stability of the catheter. In the present embodiment, it is possible for the structural elements on the outside to be directly opposite the structural elements on the inside. It is also possible for the structural elements on the outside to be offset from the structural elements on the inside. For example, it may be that the structural elements are disposed along a first partial segment of the distal end segment on the outside of the body and along a second partial segment of the distal end segment on an inside of the body. Here, too, it is preferable that there is no insulating layer between the conductive structure and the tumour tissue or vessel.

Another way to improve the stability of the catheter is to embed the conductive structure partially or completely in a wall of the cylindrical body on the outside and/or inside.

It is preferable that at least the first and second structural elements of the conductive structure are disposed parallel to a longitudinal axis of the cylindrical body. The structural elements are preferably disposed in strips along the distal end section of the cylindrical body, the strips preferably being of equal length. However, the strips may also have different lengths. It is preferable that the strips form straight lines, but said strips may also be wavy.

In an alternative embodiment, it is preferred that at least the first and second structural elements of the conductive structure are disposed elliptically along the distal end segment of the cylindrical body. The structural elements therefore form closed, oval curves winding around the cylindrical body. It is also preferable that more than two, for example three or four elliptical structural elements are provided. The elliptical structural elements are preferably disposed at equal distances from each other. However, it is also possible for the elliptical structural elements to be disposed at different distances from each other.

In a further, alternative embodiment, it is preferable that at least the first and second structural elements of the conductive structure are disposed in a ring shape and coaxially to a longitudinal axis of the cylindrical body. The ring-shaped structural elements are preferably disposed at equal distances from each other. However, it is also possible to provide different distances between the structural elements.

In a further, alternative embodiment, the conductive structure is preferably disposed in the form of a net along the end section of the cylindrical body. To this end, at least the first and second structural elements wind helically around the cylindrical body, the first structural element winding around the cylindrical body in a left-hand direction and the second structural element winding around the cylindrical body in a right-hand direction. Furthermore, the first and second structural elements preferably wind around the body at a constant pitch. It is possible for the structural elements to be continuous or interrupted in sections.

For the present embodiment, it is preferable that the structural elements are insulated from each other by an insulating intermediate layer of an insulating material, at least at the interfaces where the two structural elements intersect.

In addition, further arrangement patterns of the structural elements of the conductive structure along the end section of the cylindrical body are possible, such as circular, semi-circular, corrugated, or serrated arrangement patterns. Furthermore, the arrangement patterns may be combined with each other as desired. The different arrangement patterns of the structural elements enable electric fields having different properties and spatial expansions. The different arrangements of the structural elements therefore make it possible to generate electric fields matched to the spatial extent of the tumour tissue to be treated.

It is preferred that the electrically conductive structure generates pulsed electric fields when a voltage is applied, inducing reversible pore formation in the cell membranes of the tumour tissue. In order for the cell membranes to become reversibly permeable, the pulsed electric fields generated must have electric field strengths exceeding certain threshold values. The threshold values depend in particular on the electrical properties of the target tissue, the pulse width used, and the number of pulses. The threshold values should also be set so that reversible pore formation preferably occurs exclusively in the target tissue and the neighbouring structures remain undamaged. The pulsed electric fields (PEF) generated are preferably characterised by high electric field strengths and a short duration. For reversible electroporation, “millisecond pulsed electric fields” (msPEF), “microsecond pulsed electric fields” (μsPEF), and/or “nanosecond pulsed electric fields” (nsPEF) are preferred. Furthermore, pulsed electric fields having a pulse duration in the range of seconds, picoseconds, and/or femtoseconds are preferred. The reversible pore formation in the cell membranes also causes a temporary permeability for chemical and/or biological therapeutic agents, so that the same can diffuse into the cell interior. For electrochemotherapy of skin tumours, pulse protocols of 8 pulses, each having a pulse width of 100 μs, are known from the state of the art, whereby the field strength to be achieved is preferably in the range of 1 to 1.4 kV/cm (Andreas Ritter. “Strategien und Elektrodendesign für die patientenindividuelle tumortherapeutische Anwendung der Elektroporation” [“Strategies and electrode design for the patient-specific tumour therapeutic application of electroporation”]. Doctoral thesis. RWTH Aachen University, Faculty of Electrical Engineering and Information Technology, May 2017).

It is particularly preferred that the electrically conductive structure generates pulsed electric fields when a voltage is applied, which induce reversible pore formation in cell membranes of the tumour tissue for introducing calcium. It has been shown that introducing calcium into cells is a safe and efficient therapeutic procedure for treating tumour tissue. A particular advantage of calcium electroporation is that the healthy surrounding tissue or structure is less affected by the calcium than the tumour tissue itself. One reason for this could be that healthy cells, in contrast to tumour cells, can rebuild their membrane more effectively and more quickly after electroporation, which means that the permeability of healthy cells is shortened and weakened compared to tumour cells. In addition, it has been shown that healthy cells, in contrast to tumour cells, break down the introduced calcium better and can return more quickly to an intracellular calcium level corresponding to the calcium level of untreated cells.

Alternatively, it is preferred that the electrically conductive structure generates electric fields when a voltage is applied, causing irreversible pore formation in the cell membranes of the tumour tissue. For this purpose, the electric field strengths of the pulsed electric fields generated must also exceed threshold values, the threshold values for irreversible electroporation being higher than for reversible electroporation. The threshold values for irreversible electroporation depend in particular on the electrical properties of the target tissue, the pulse width used, and the number of pulses. For irreversible electroporation, “millisecond pulsed electric fields” (msPEF), “microsecond pulsed electric fields” (μsPEF), and/or “nanosecond pulsed electric fields” (nsPEF) are preferred. Furthermore, pulsed electric fields having a pulse duration in the range of seconds, picoseconds, and/or femtoseconds are preferred. For irreversible electroporation in liver tumours, pulse protocols of 90 pulses, each having a pulse width in the range of 50 μs to 100 μs, are known from the state of the art, whereby the field strength to be achieved in irreversible electroporation is preferably at least 1 kV/cm (Andreas Ritter. “Strategien und Elektrodendesign für die patientenindividuelle tumortherapeutische Anwendung der Elektroporation” [“Strategies and electrode design for the patient-specific tumour therapeutic application of electroporation”]. Doctoral thesis. RWTH Aachen University, Faculty of Electrical Engineering and Information Technology, May 2017). As a result of the irreversible cell damage, the treated tissue cells die. To prevent the surrounding structures from dying, the threshold values must be selected in such a way that the electrical properties of the surrounding structure remain unaffected.

Furthermore, it is preferred that the energy source provides an energy suitable for thermal ablation of the tumour tissue. For example, radio frequency energy or microwave energy.

The conductive structure is preferably formed partly or completely from an electrically conductive plastic. The advantages of plastics include being able to be moulded in a variety of ways, being highly resistant to chemicals, having a lower density than metal, and allowing a high degree of design freedom. Due to the simple mouldability using conventional moulding methods for plastics and the high degree of design freedom, the diverse arrangement patterns of the conductive structure along the end section of the cylindrical body are easy to implement. A conductive structure partially or completely formed from an electrically conductive plastic has the advantage over a conductive structure formed from a wire and/or a conductive metal of being significantly more flexible. This means that a conductive structure made of an electrically conductive plastic adapts better to the conditions in the body when the catheter is passed through the body. In this respect, a conductive structure made of an electrically conductive plastic can better simulate the bends and curvatures of the vessels through which the catheter is passed. This makes it much easier to guide the catheter.

The conductive structure is preferably formed partially or completely from a doped plastic. This meant that some electrons in the polymer chains of the corresponding polymers are removed (p-doping) or added (n-doping). As a result, individual free electrons remain and slide along the molecules and can thus transport the electrical charge.

Alternatively, it is preferable that the conductive structure is partially or completely formed from a plastic material mixed with electrically conductive additives. The addition of the additives must be sufficiently large so that there is a high probability of the additives coming into contact with each other and consequently forming continuous current paths. As the additive concentration increases, so does the conductivity of the polymer. The additives may be formed from any electrically conductive material. Examples include carbon, conductive carbon black, aluminium, or stainless steel.

Preferably, the conductive structure is formed partially or completely from a fibre-plastic composite. With fibres as additives, disposed randomly, there is a high probability of contact even at low concentrations. It is particularly preferred that the conductive structure is partially or completely formed from a carbon fibre-reinforced plastic. However, other additives in the fibre structure are also conceivable, for example carbon fibres. A combination of different additives in the plastic is also possible, whereby additives having a fibre structure and additives having no fibre structure may also be combined.

In a further, preferred embodiment, the catheter comprises a balloon, wherein in the present embodiment the conductive structure extends at least in sections along the balloon. The conductive structure, comprising at least two structural elements, may also be disposed on an outer side, on an inner side, or both on an outer side and on an inner side of the balloon. It may also be partially or fully embedded in a wall of the balloon. Furthermore, the conductive structure may be disposed along the balloon according to the different arrangement patterns described above, e.g. strip-shaped, net-shaped, or ring-shaped, so that reference is made to the previous description. In the embodiment of the catheter having a balloon, it is also preferred that the body of the catheter has a further, fluid-conducting lumen provided for expanding the balloon by means of pressurised air or liquid.

Another object of the invention is a method for locally treating tumour tissue in intravascular and intraluminal spaces using the catheter described above.

First, a percutaneous access is placed and secured. For example, the access can be made to the bile duct system, to a blood vessel, or to another anatomical hollow organ or vascular system. Alternatively, it is possible to gain access via natural body openings, e.g. via the oesophagus or the bowel. The patient may be awake or sedated during placement and securing of the access. It is preferable that at least the area of the percutaneous access is locally anaesthetised. It is also preferable that the percutaneous access is placed using the Seldinger technique. For the Seldinger technique, a wire is first inserted via a puncture cannula and then the puncture cannula is replaced with an insertion aid (sheath) via said wire.

The method also includes the step of localising the target lesion using imaging, where the target lesion is an area of tumour tissue to be treated using the catheter. Possible imaging procedures are X-ray, computer tomography (CT), magnetic resonance imaging (MRI), sonography, or endoscopy.

The target lesion is also visualised using a contrast agent. If the target lesion is located in the bile duct system, contrast agent may be injected into the bile ducts. The target lesion is demarcated from the surrounding structures as a gap in contrast agent and thus becomes visible.

The procedure also includes the step of image-guided guiding of the catheter via the guide wire to the target lesion. The distal end section of the catheter, comprising the conductive structure, can be placed precisely at the target lesion in this way.

In a preferred step, the method comprises applying physical and/or chemical and/or biological measures. For this purpose, pulsed electric fields are generated by means of the conductive structure and physically affect the target lesion in such a way that pores form in the cell membranes of the target lesion, wherein the pore formation may be reversible or irreversible. An irreversible pore formation results from an irreversible electroporation, A reversible pore formation results from a reversible electroporation, wherein the pores created are small enough to close themselves again on the one hand and large enough to allow molecules of a chemical and/or biological therapeutic agent to pass through on the other. Chemical and/or biological therapeutic agents may be introduced separately and preferably independently into the porous target lesion via the second and third lumen and, if necessary, via further lumens. Possible therapeutic agents include cytostatics, saline solutions, antibiotics, calcium, immunosuppressive agents, and/or other agents bringing about cell death. The method particularly preferably comprises introducing calcium into the porous target lesion. It is particularly preferred that calcium is introduced into the porous target lesion in an amount of 2.5 mM to 20 mM. Amounts above 20 mM are also preferred. Even introducing a quantity of 2.5 mM calcium into the target lesion can cause the cells to die after reversible electroporation or reduce the survivability of the cells to a range of 0% to 3%. In a further, preferred embodiment of the procedure, heat is used to act on the target lesion.

Once the target lesion has been treated, the catheter is removed from the body in a final step.

In a preferred refinement of the method, the treatment effect is visualised using imaging techniques. Possible imaging procedures are also X-ray, CT, MRI, sonography, and/or endoscopy. It is preferable that the same imaging method is used to visualise the treatment effect as for localising the target lesion, so that better comparability can be achieved before and after treatment of the target lesion.

In a further, preferred refinement of the procedure, the percutaneous access for repeated treatment of the tumour tissue is secured by means of the catheter and the procedure carried out. Preferably, percutaneous access is secured using a wire or a sheath. It is also preferable that the treatment effect is visualised using imaging techniques after the repeated treatments as well.

A further object of the invention is a catheter system comprising the described catheter and a control unit for controlling the energy source, wherein the catheter system is configured to carry out the method described above. The control unit may control the energy source in such a way that variable parameter combinations of the parameters voltage, current, pulse duration, pulse frequency, and/or number of pulses are possible, so that the conductive structure can consequently generate electric fields having different properties, particularly preferably having different field strengths.

Another object of the invention is a method for creating a three-dimensional map of an organ having a tumour. The method comprises measuring individual, tissue-specific electrical properties of the organ, preferably non-invasively, particularly preferably by electrical impedance tomography (EIT) or magnetic resonance imaging (MRI). The procedure also involves segmenting the tumour and the tissue surrounding the tumour. The procedure also includes the creation of three-dimensional maps, taking into account the segmentation and the individual, tissue-specific electrical properties of the organ.

It is preferred that the control unit controls the energy source taking into account the three-dimensional map of the organ, whereby the energy source is preferably designed having one or more or all of the features as above. Preferably, the control unit is configured to control the energy source to generate electric fields configured to the individual, tissue-specific electrical properties of the organ. Preferably, the control unit is disposed to control the energy source so as to provide a first potential at a first structural element of a catheter and to provide a second potential at a second structural element of the catheter, wherein the first potential is higher or lower than the second potential, wherein the catheter is preferably designed having one or more or all of the features as above. Furthermore, the control unit is preferably configured to control the energy source in such a way that the energy source provides different potentials at the first, the second, and/or the other structural elements of the catheter. In this way, different voltages and thus different fields can be generated between the structural elements.

Embodiments of the invention are described below with reference to the drawings. Said drawings are not necessarily intended to depict the embodiments to scale; rather, the drawings are shown in schematic and/or slightly distorted form for explanatory purposes. With respect to supplements to the teachings directly discernible from the drawings, reference is made to the applicable prior art. It must be taken into account that various modifications and changes relating to the shape and detail of an embodiment may be made without deviating from the general idea of the invention. The features of the invention disclosed in the description, in the drawings, and in the claims may be essential to the refinement of the invention individually and in any arbitrary combination. In addition, all combinations of at least two of the features disclosed in the description, the drawings, and/or the claims fall within the scope of the invention. The general idea of the invention is not limited to the precise form or the detail of the preferred embodiments shown and described below or limited to a subject-matter that would be limited in comparison with the subject-matter claimed in the claims. In the case of the specified measurement ranges, values lying within the stated limits should also be disclosed as limit values and be able to be used and claimed as required. For simplicity, identical reference numerals are used below for identical or similar parts or parts having identical or similar functions.

Further advantages, features, and details of the invention result from the below description of the preferred embodiments and from the drawings, which show:

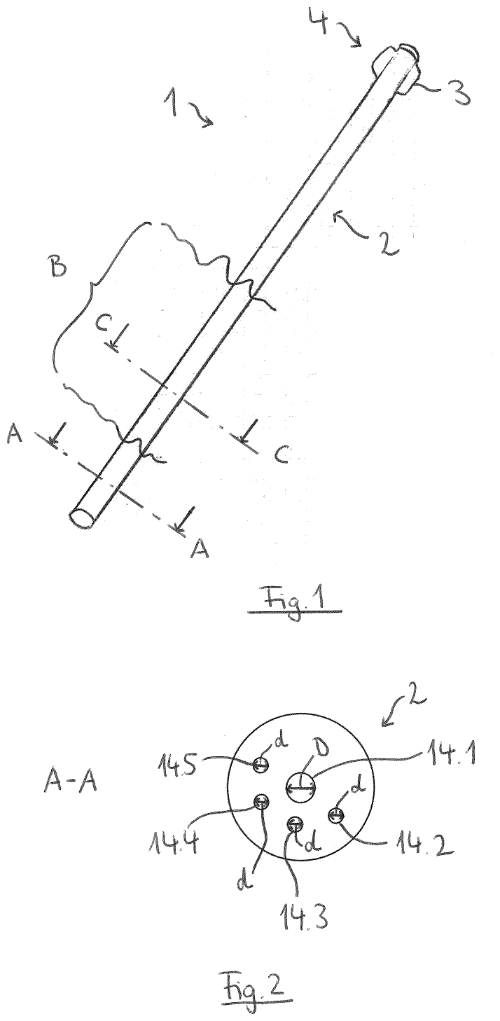

FIG. 1 a schematic representation of a catheter having a cylindrical body and a Luer connection;

FIG. 2 a cross-section through the catheter along A-A as shown in FIG. 1;

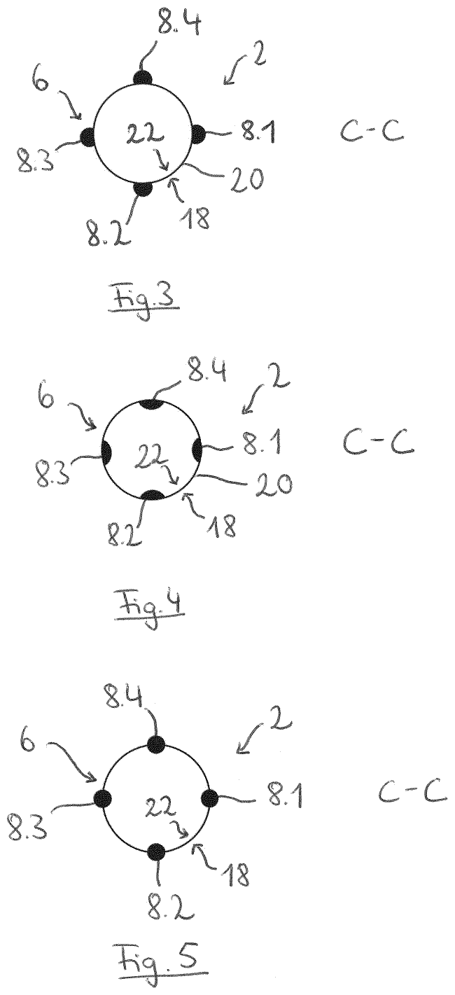

FIG. 3 an arrangement of the conductive structure on the body according to a first arrangement;

FIG. 4 an arrangement of the conductive structure on the body according to a second arrangement;

FIG. 5 an arrangement of the conductive structure on the body according to a third arrangement;

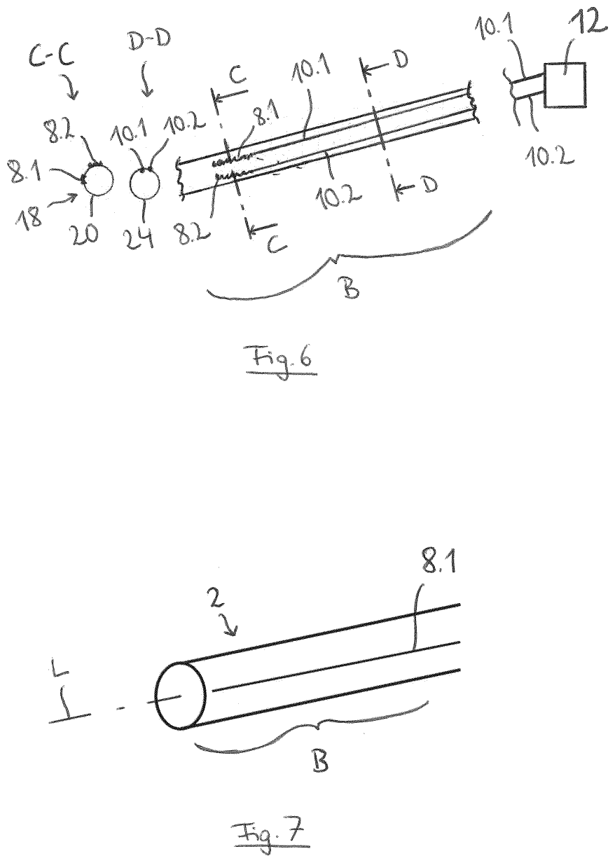

FIG. 6 a first schematic representation of the distal end section B according to a first embodiment example;

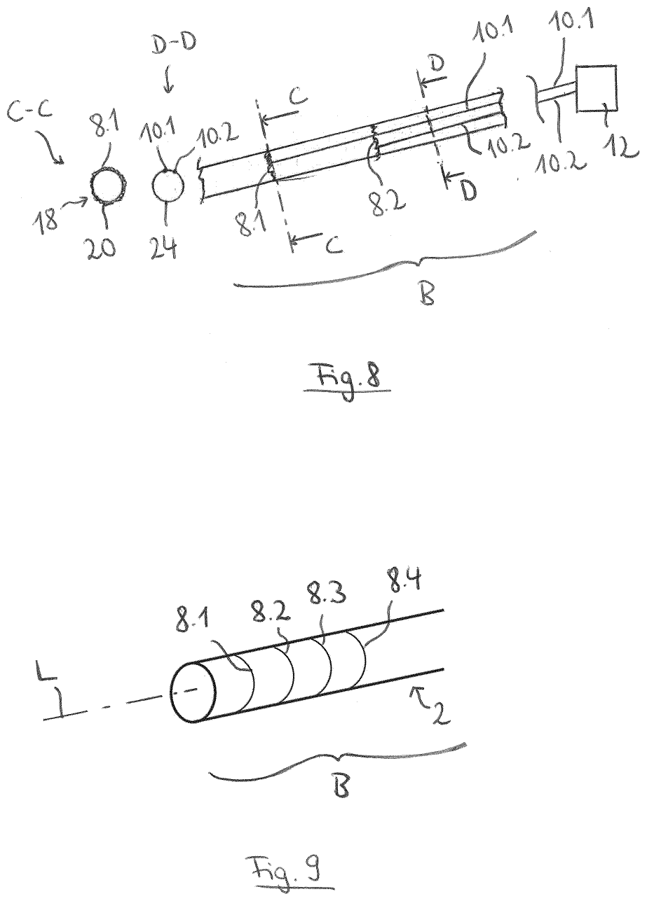

FIG. 7 a second schematic representation of the distal end section B according to the first embodiment example;

FIG. 8 a first schematic representation of the distal end section B according to a second embodiment example;

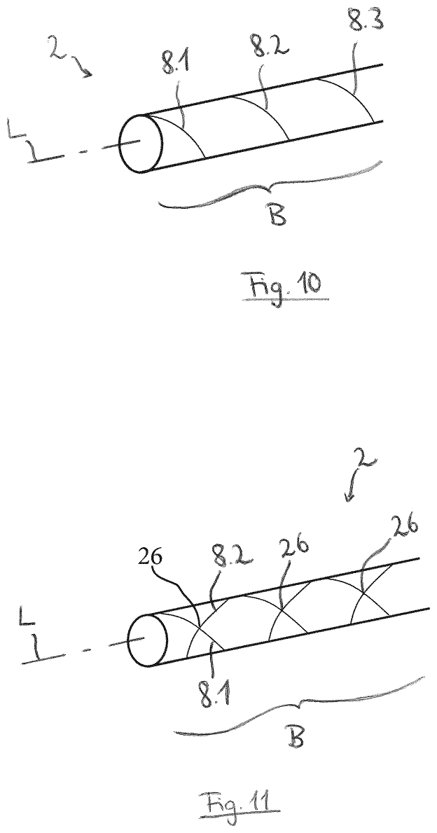

FIG. 9 a second schematic representation of the distal end section B according to the second embodiment example;

FIG. 10 a schematic representation of the distal end section B according to a third embodiment example;

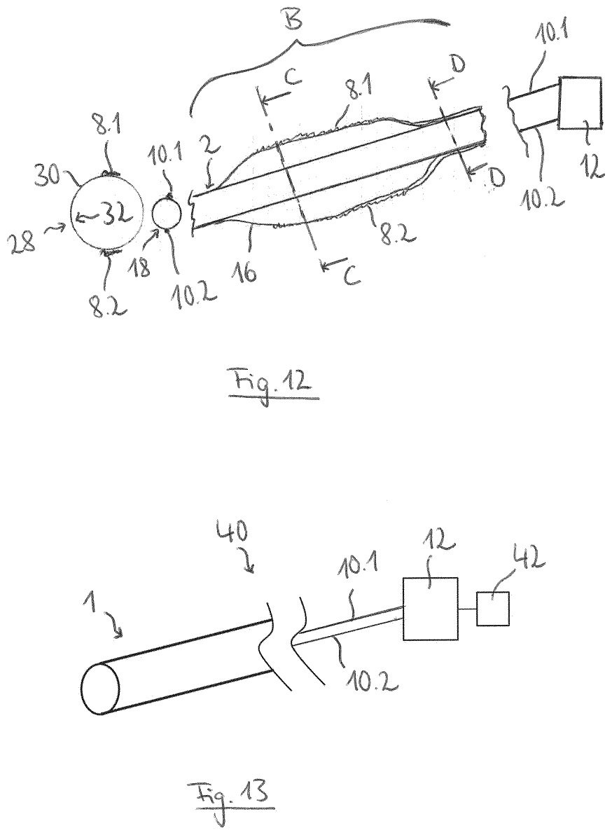

FIG. 11 a schematic representation of the distal end section B according to a fourth embodiment example;

FIG. 12 a schematic representation of the distal end section B having an expandable balloon; and

FIG. 13 a schematic representation of the catheter system.

FIG. 1 shows a catheter 1 having a cylindrical body 2 and a Luer connector 3 disposed on a proximal section 4 of the cylindrical body 2. The Luer connection 3 is a standardised connection system by means of which the cylindrical body 2 of the catheter 1 may be connected to other tubes or hoses. The length of the Luer connection 3 is variable and the diameter of the Luer connection 3 is preferably in the range from 3 mm to 8 mm.

The cylindrical body 2 of the catheter 1 is flexible so that said catheter may be easily guided to the target lesion. Furthermore, the catheter 1 is configured to be torsionally stable and tension-resistant. Said catheter is made of a thermoplastic, elastomer, or silicone and has radiopaque features so that the catheter 1 is clearly recognisable during image-guided guiding. The length of the cylindrical body 2 is preferably selected so that the tumour tissue to be treated is reached.

The cylindrical body 2 has a distal end section B having a conductive structure 6 (see FIG. 6-12). The conductive structure 6 comprises at least a first and a second structural element 8.1, 8.2, each of which is connected to an energy source 12 via an electrical supply line 10.1, 10.2. When a voltage is applied to the structural elements 8.1, 8.2, an electric field is generated. The energy source 12 can provide different voltages, currents, pulse durations and frequencies as well as a variable number of pulses via the supply lines 10.1, 10.2 to the structural elements 8.1, 8.2. Pulsed electric fields may thus be generated for causing reversible electroporation, irreversible electroporation, or heat ablation (see FIGS. 6, 8, 12). It is preferable that the energy source 12 is configured to generate different electric fields along the catheter 1. The energy source 12 preferably provides different potentials at the structural elements for this purpose. Electric fields having different field strengths are thus formed between the structural elements, for example between the structural elements 8.1 and 8.2 and the structural elements 8.3 and 8.4 (see FIG. 9), whereby the higher the voltage between the structural elements, the higher the electric field strength. FIG. 2 shows a cross-section A-A through the cylindrical body 2 of the catheter 1. The cross-section A-A shows that the cylindrical body 2 has a first lumen 14.1 disposed in the centre, and four further lumens 14.2-14.5 disposed around the first lumen 14.1. In the embodiment example shown, the diameter D of the first lumen 14.1 is larger than the diameters d of the other lumens 14.2-14.5. The diameters of the lumens may also be the same size or different from each other. The arrangement and the number of lumens are also not limited to the design example shown.

The first lumen 14.1 is preferably suitable for receiving a guide wire (not shown) and will therefore also be referred to as a guide channel. The four other lumens 14.2-14.5 may be described as functional channels. The functional channels 14.2-14.5 are preferably suitable for conveying chemical and biological therapeutic agents as well as other fluids. The chemical and biological therapeutic agents may be conveyed together in one functional channel or separately in different functional channels. In the event that the catheter 1 has an expandable balloon 16, compressed air or liquid may also be conveyed in a further functional channel to expand the balloon. Rinsing solutions may also be conveyed in lumens 14.2-14.5.

The functional channels 14.2-14.5 for conveying chemical and/or biological therapeutic agents and thus for introducing the therapeutic agents into the tumour tissue open into openings (not shown) positioned anywhere along the cylindrical body 2. Preferably, the functional channels 14.2-14.5 for conveying chemical and/or biological therapeutic agents open into openings disposed at the distal end segment B of the cylindrical body 2. This enables the targeted introduction of therapeutic agents into the physically treated tumour tissue. It is particularly preferable that at least one of the functional channels 14.2-14.5 is configured to convey calcium.

FIG. 3 shows a first cross-section along section C-C as shown in FIG. 1 through the distal end section B of the cylindrical body 2. According to said first arrangement, the conductive structure 6 is disposed on an outer surface 20 of the cylindrical body 2. In said arrangement, the conductive structure 6 comprises four structural elements 8.1-8.4 evenly spaced around the cylindrical body 2.

The structural elements 8.1-8.4 can be in the form of a wire having a partially circular cross-section, wherein the wire is made of any electrically conductive material such as stainless steel, copper, gold, or silver, or another conductive metal or alloys thereof. The wire may be connected to the cylindrical body 2 using a suitable joining process, e.g. glued or soldered to the cylindrical body 2 or partially overmoulded by the material of the body.

However, the structural elements 8.1-8.4 are particularly preferably formed from an elongated, flexible conductive plastic. Structural elements 8.1-8.4 made of a conductive plastic increase the flexibility of the catheter. The structural elements 8.1-8.4 made of conductive plastic may be joined to the cylindrical body 2 using a suitable joining process or moulded directly together with the cylindrical body 2, e.g. co-extruded.

In said first arrangement according to FIG. 3, the structural elements 8.1-8.4 have radially protruding profiles so that the structural elements 8.1-8.4 can press sufficiently well into the tumour tissue or vessel. However, structural elements having significantly flatter, square, or triangular profiles are also possible and preferred.

FIG. 4 shows a further cross-section C-C through the distal end section B of the cylindrical body 2. According to said second arrangement, the conductive structure 6 is disposed on an inner side 22 of the cylindrical body 2. In this arrangement, the conductive structure 6 comprises four structural elements 8.1-8.4 with comparatively flat, inwardly rounded profiles. The cylindrical body 2 is uniformly round on the outside. No insulating layer is provided between the conductive structure 6 and an outer side 18 of the cylindrical body 2, so that conductivity to the outside is ensured.

FIG. 5 shows a further cross-section C-C through the distal end section B of the cylindrical body 2. In said third arrangement, the conductive structure 6 is disposed on both the outer side 18 and the inner side 22 of the cylindrical body 2. The structural elements on the outer side 18 are directly opposite those on the inner side 22. In said third arrangement, the opposing structural elements form a closed unit. This means that there is no separating layer between the opposing structural elements, but that said elements are moulded as a composite. The resulting four structural elements 8.1-8.4 are circular in shape and partially embedded in a wall 24 of the cylindrical body 2. The stability of the catheter 1 may be improved by said structural elements 8.1-8.4 moulded both outwards and inwards.

FIG. 6 shows a first schematic representation of the distal end section B according to a first embodiment example. The distal end section B according to the first embodiment example has a conductive structure 6 having two structural elements 8.1, 8.2, each connected to the energy source 12 via an electrical supply line 10.1, 10.2. The two strip-shaped structural elements 8.1, 8.2 run parallel to each other and parallel to a longitudinal axis L of the cylindrical body 2 (see FIG. 7). The two electrical supply lines 10.1, 10.2 also run parallel to each other and parallel to the longitudinal axis L of the cylindrical body 2. The two electrical supply lines 10.1, 10.2 are made of any electrically conductive material such as stainless steel, copper, gold, silver, or conductive plastic. In addition, said lines are isolated from each other to ensure that the energy from the energy source 12 is channelled undisturbed to the structural elements 8.1, 8.2.

FIG. 6 also shows the cross-section C-C in the area of the conductive structure 6 and the cross-section D-D in the area of the electrical supply lines 10.1, 10.2. According to the first embodiment example, the conductive structure 6 is disposed on the outer surface 20 of the cylindrical body 2 (see cross-section C-C, FIG. 6). The arrangement of the conductive structure 6 therefore corresponds to the arrangement shown in FIG. 3. However, it is alternatively possible for the conductive structure 6 to be disposed on the inner side 22 of the cylindrical body 2 as shown in FIG. 4 or both on the inner side 22 and on the outer side 18 of the cylindrical body 2 as shown in FIG. 5. The electrical supply lines 10.1, 10.2 are partially embedded in the wall 24 of the cylindrical body 2. The wall 24 of the cylindrical body 2 is preferably made of an insulating plastic and forms an insulating layer between the electrical supply lines 10.1, 10.2 (see cross-section D-D, FIG. 6). However, the electrical supply lines 10.1, 10.2 may also be completely embedded in the insulating wall 24 of the cylindrical body 2.

FIG. 7 shows a second schematic representation of the distal end section B according to the first embodiment example in FIG. 6. In this view, only one structural element 8.1 of the conductive structure 6 is shown. The structural element 8.1 also extends parallel to the longitudinal axis L of the cylindrical body 2 along the distal end section B and forms a straight line. The length of the strip-shaped structural element 8.1 should preferably be selected depending on the tumour tissue to be treated.

FIG. 8 shows a first schematic representation of the distal end section B according to a second embodiment example. The distal end section B according to the second embodiment example also has a conductive structure 6 having two structural elements 8.1, 8.2, each connected to an energy source 12 via an electrical supply line 10.1, 10.2. The two structural elements 8.1, 8.2 are ring-shaped and disposed coaxially to the longitudinal axis L of the cylindrical body 2 (see FIG. 9). The two electrical supply lines 10.1, 10.2 run parallel to each other and parallel to the longitudinal axis L of the cylindrical body 2.

FIG. 8 also shows the cross-section C-C in the area of the conductive structure 6 and the cross-section along the line D-D according to FIG. 6 in the area of the electrical supply lines 10.1, 10.2. The conductive structure 6 is disposed on the outer surface 20 of the cylindrical body 2 (see cross-section C-C, FIG. 8). The arrangement of the conductive structure 6 therefore also corresponds to the arrangement shown in FIG. 3. Here, too, it is alternatively possible for the conductive structure 6 to be disposed on the inside 22 of the cylindrical body 2 as shown in FIG. 4 or both on the inside 22 and on the outside 20 of the cylindrical body 2 as shown in FIG. 5. The electrical supply lines 10.1, 10.2 are partially embedded in the wall 24 of the cylindrical body 2 and are insulated from each other. The wall 24 also forms the insulating layer between the two electrical supply lines 10.1, 10.2 (see cross-section D-D, FIG. 8). However, the electrical supply lines 10.1, 10.2 may also be completely embedded in the wall 24 of the cylindrical body 2.

FIG. 9 shows a second schematic representation of the distal end section B according to the second embodiment example having four structural elements 8.1-8.4 disposed annularly and coaxially to the longitudinal axis L of the cylindrical body 2. The structural elements 8.1-8.4 are evenly spaced along the distal end section B. The electrical supply lines 10.1-10.4 by means of which the structural elements 8.1-8.4 are connected to the energy source 12 are not shown in FIG. 9.

FIG. 10 shows a schematic representation of the distal end section B according to a third embodiment example. The distal end section B has three structural elements 8.1-8.3, which are elliptical in shape. The structural elements 8.1-8.3 therefore form closed, oval curves that wind around the cylindrical body 2. The three structural elements 8.1-8.3 are evenly spaced apart and are also each connected to the energy source 12 via an electrical supply line 10.1-10.2 (not shown). The elliptical structural elements 8.1-8.3 are disposed on the outside 18 of the cylindrical body 2 as shown in FIG. 10. However, said elements may also be disposed on the inside 22 or both on the inside 22 and on the outside 18 of the cylindrical body 2.

FIG. 11 shows a schematic representation of the distal end section B according to a fourth embodiment example. In said fourth embodiment example, the conductive structure 6 is disposed in a net shape along the distal end section B. A first and a second structural element 8.1, 8.2 wind helically around the cylindrical body 2, wherein the first structural element 8.1 winds clockwise and the second structural element 8.2 anti-clockwise around the cylindrical body 2. The structural elements 8.1, 8.2 wind around the cylindrical body 2 at a constant and equal pitch and, when viewed together, form the shape of a net. At the interfaces 26, where the two structural elements 8.1, 8.2 intersect, the structural elements 8.1, 8.2 are insulated from each other. An insulating intermediate layer made of plastic is provided for this purpose, for example.

FIG. 12 shows a schematic representation of the distal end section B according to a fourth embodiment example. In said fourth embodiment example, the catheter 1 further comprises an expandable balloon 16. The conductive structure 6 is now no longer disposed on the cylindrical body 2, but on the expandable balloon 16. In said embodiment example, the conductive structure 6 comprises two structural elements 8.1, 8.2 disposed opposite each other and extending in the form of elongated strips along an outer balloon surface 30 (see FIG. 11, cross-section C-C). The structural elements 8.1, 8.2 may also be disposed on the balloon 16 in different ways according to the arrangement patterns already described, i.e. elliptical, ring-shaped, and/or net-shaped. An arrangement of the conductive structure 6 on a balloon inner side 32 or on both a balloon inner side 32 and a balloon outer side 28 is also possible and preferred.

The structural elements 8.1, 8.2 are each connected to the energy source 12 by means of an electrical supply line 10.1, 10.2. It can be seen from the cross-section D-D of FIG. 12 that the supply lines 10.1, 10.2 are disposed on the outside 18 of the cylindrical body 2.

FIG. 13 shows a schematic representation of a catheter system 40. The catheter system 40 comprises the catheter 1 together with the energy source 12 and a control unit 42 connected to the energy source 12. The catheter 12 is only shown schematically and partially cut off in FIG. 13. The at least first and at least second supply lines 10.1, 10.2 are coupled to the energy source 12 so that the energy source can provide energy to the conductive structure (not shown in FIG. 13).

The control unit 42 is configured to control the energy source 12. Variable parameter combinations of the parameters voltage, current, pulse duration, pulse frequency, and number of pulses may be controlled to generate electric fields with different properties. In this way, pulsed electric fields may be generated, resulting in reversible or irreversible electroporation. Taking into account the specific electrical properties, i.e. the permittivity and electrical conductivity of the tumour tissue and the adjacent tissue, the parameters may also be controlled in such a way that only the tumour tissue is exposed to electroporation. The surrounding tissue may thus retain the structure thereof. The control unit 42 is preferably configured to control the energy source 12 to generate different electric fields between the structural elements 8.1-8.4. It is preferred that the control unit 42 controls the energy source 12 taking into account the tissue-specific electrical properties of the tumour tissue.

### LIST OF REFERENCE NUMERALS

1 Catheter
2 Cylindrical body
3 Luer connection
4 Proximal section of the catheter
6 Conductive structure
8.1-8.4 First, second, third and fourth structural element
10.1-10.4 First, second, third and fourth supply line
12 Energy source
14.1 First lumen
14.2-14.5 Second, third, fourth, and fifth lumen
16 Balloon
18 Exterior of the cylindrical body
20 Outer surface of the cylindrical body
22 Interior of the cylindrical body
24 Wall of the cylindrical body
26 Interface
28 Balloon exterior
30 Balloon surface
32 Balloon interior
40 Catheter system
42 Control unit
B Distal end section of the catheter
D Diameter of the first lumen
d Diameter of the second, third, fourth, and fifth lumen
L Longitudinal axis of the cylindrical body

1. A catheter for locally treating a tissue in intravascular and intraluminal spaces, the catheter comprising
a flexible, cylindrical body having a first lumen for receiving a guide wire for controlling the catheter, a second lumen, and at least one third lumen for separately introducing chemical and/or biological therapeutic agents into the tissue, wherein the cylindrical body has radiopaque features,
a conductive structure for generating pulsed electric fields, wherein the conductive structure comprises at least a first structural element and a second structural element and extends along a distal end segment of the cylindrical body, and
a first electrical supply line for connecting the first structural element to an energy source and a second electrical supply line for connecting the second structural element to the energy source, wherein the first and second supply lines are insulated from each other.

2. The catheter according to claim 1, wherein the conductive structure is disposed on an outer surface of the cylindrical body.

3. The catheter according to claim 1, wherein the conductive structure is disposed on an inner side of the cylindrical body.

4. The catheter according to claim 3, wherein the conductive structure is disposed both on an outer side and on the inner side of the cylindrical body.

5. The catheter according to claim 1, wherein at least the first structural element and the second structural element of the conductive structure are disposed parallel to a longitudinal axis of the cylindrical body.

6. The catheter according to claim 1, wherein at least the first structural element and second structural element are disposed elliptically along the distal end segment of the cylindrical body.

7. The catheter according to claim 1, wherein at least the first structural element and the second structural element of the conductive structure are disposed in an annular and coaxial manner with respect to a longitudinal axis of the cylindrical body.

8. The catheter according to claim 1, wherein the conductive structure is disposed in a net-shaped manner along the distal end segment of the cylindrical body.

9. The catheter according to claim 1, wherein the conductive structure generates pulsed electric fields when a voltage is applied, causing reversible pore formation in cell membranes of the tissue.

10. The catheter according to claim 1, wherein the conductive structure generates pulsed electric fields when a voltage is applied, causing irreversible pore formation in cell membranes of the tissue.

11. The catheter according to claim 1, wherein the conductive structure is partially or completely formed from an electrically conductive plastic.

12. The catheter according to claim 11, wherein the conductive structure is partially or completely formed from a doped plastic.

13. The catheter according to claim 11, wherein the conductive structure is partially or completely formed from a plastic material mixed with electrically conductive additives.

14. The catheter according to claim 13, wherein the conductive structure is partially or completely formed from a fibre-plastic composite.

15. The catheter according to claim 14, wherein the conductive structure is partially or completely formed from a carbon fibre-reinforced plastic.

16. The catheter according to claim 1, further comprising an expandable balloon, wherein the conductive structure extends at least in sections along the balloon.

17. A method for locally treating tissue, in particular tumour tissue or other undesirable tissue types, in intravascular and intraluminal spaces with a catheter, the method comprising:
placing and securing a percutaneous access;
imaging localisation of a target lesion;
visualising the target lesion by a contrast agent;
image-guided guiding of the catheter to the target lesion via a guide wire;
applying physical and/or chemical and/or biological measures; and
removing the catheter .

18. The method according to claim 17, further comprising visualising a treatment effect by imaging techniques.

19. The method according to claim 17, further comprising securing the percutaneous access for repeated treatment of the tissue by means of the catheter (1).

20. A catheter system (40) comprising a catheter (1) according to claim 1, wherein the catheter system (40) further comprises a control unit (42) for controlling the energy source (12).
